(19) United States
(12) Patent Application Publication
(10) Pub. No.: US 2022/0415459 A1
(43) Pub. Date: Dec. 29, 2022

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventor: Akimichi ICHINOSE, Tokyo (JP)

(73) Assignee: FUJIFILM Corporation, Tokyo (JP)

(21) Appl. No.: 17/900,827

(22) Filed: Aug. 31, 2022

Related U.S. Application Data
(63) Continuation of application No. PCT/JP2021/008222, filed on Mar. 3, 2021.

(30) Foreign Application Priority Data
Mar. 3, 2020 (JP) 2020-036290

(51) Int. Cl. G16H 10/60; G16H 30/40; G06T 7/00

(52) U.S. Cl. CPC G16H 10/60; G16H 30/40; G06T 7/0016

(57) ABSTRACT

An information processing apparatus including at least one processor, wherein the at least one processor is configured to: derive a property score indicating a prominence of a property for each of predetermined property items from at least one image; and derive, for each of the property items, a description score indicating a degree of recommendation for including a description regarding the property item in a document.

### CROSS REFERENCE TO RELATED APPLICATIONS

The present application is a Continuation of PCT International Application No. PCT/JP2021/008222, filed on Mar. 3, 2021, which claims priority to Japanese Patent Application No. 2020-036290, filed on Mar. 3, 2020. Each application above is hereby expressly incorporated by reference, in its entirety, into the present application.

### BACKGROUND

### Technical Field

The present disclosure relates to an information processing apparatus, an information processing method, and an information processing program for supporting creation of documents such as interpretation reports.

### Related Art

In recent years, advances in medical devices, such as computed tomography (CT) apparatuses and magnetic resonance imaging (MM) apparatuses, have enabled image diagnosis using high-resolution medical images with higher quality. In particular, since a region of a lesion can be accurately specified by image diagnosis using CT images, MRI images, and the like, appropriate treatment is being performed based on the specified result.

In addition, image diagnosis is made by analyzing a medical image via computer-aided diagnosis (CAD) using a discriminator in which learning is performed by deep learning or the like, and discriminating properties such as the shape, density, position, and size of a structure of interest such as a lesion included in the medical image. The analysis result obtained in this way is saved in a database in association with examination information, such as a patient name, gender, age, and an imaging apparatus which has acquired a medical image. The medical image and the analysis result are transmitted to a terminal of a radiologist who interprets the medical images. The radiologist interprets the medical image by referring to the distributed medical image and analysis result and creates an interpretation report, in his or her own interpretation terminal.

Meanwhile, with the improvement of the performance of the CT apparatus and the MRI apparatus described above, the number of medical images to be interpreted is increasing. Therefore, in order to reduce the burden of the interpretation work of a radiologist, various methods have been proposed to support the creation of medical documents such as interpretation reports.

For example, JP2010-167144A discloses a method of analyzing the size or the like of a nodule from the position information of the nodule in a medical image input by a radiologist, and pasting the analyzed information on the nodule together with the medical image on an interpretation report creation screen. Further, JP2017-191520A discloses that, in a case where candidates for findings such as nodular lesions and emphysema are displayed and selected by a user, the number of times or frequency of selection of each finding is stored, and the display order of the candidate for findings is determined based on the number of times or frequency of selection.

In the techniques described in JP2010-167144A and JP2017-191520A, information on the properties of structures of interest such as lesions included in medical images cannot be presented without relying on an input operation by a radiologist. Therefore, it is not sufficient to support the creation of documents such as interpretation reports.

### SUMMARY

The present disclosure provides an information processing apparatus, an information processing method, and an information processing program capable of supporting creation of documents such as interpretation reports.

According to a first aspect of the present disclosure, there is provided an information processing apparatus comprising at least one processor, in which the processor is configured to derive a property score indicating a prominence of a property for each of predetermined property items from at least one image, and derive, for each of the property items, a description score indicating a degree of recommendation for including a description regarding the property item in a document.

According to a second aspect of the present disclosure, in the above aspect, the processor may be configured to derive the description score based on a predetermined rule as to whether or not to include the description regarding the property item in the document.

According to a third aspect of the present disclosure, in the above aspect, the processor may be configured to derive the description score for each property item based on the property score corresponding to the property item.

According to a fourth aspect of the present disclosure, in the above aspect, the processor may be configured to derive the description score for any of the property items based on the property score derived for any of the other property items.

According to a fifth aspect of the present disclosure, in the above aspect, the processor may be configured to input the image into a trained model to derive the property score and the description score. The trained model may be a model that is trained by machine learning using a plurality of combinations of a training image, and the property score and the description score derived from the training image as training data, input the image, and output the property score and the description score.

According to a sixth aspect of the present disclosure, in the above aspect, the processor may be configured to derive the property score for each of a plurality of the images acquired at different points in time, and derive the description score for each property item.

According to a seventh aspect of the present disclosure, in the above aspect, the processor may be configured to derive the property score based on at least one of a position, type, or size of a structure included in the image.

According to an eighth aspect of the present disclosure, in the above aspect, the processor may generate a character string related to the image based on the description score, and perform control such that the character string is displayed on a display.

According to a ninth aspect of the present disclosure, in the above aspect, the processor may be configured to generate a character string related to a predetermined number of the property items selected in an order of the description scores.

According to a tenth aspect of the present disclosure, there is provided an information processing method, comprising: deriving a property score indicating a prominence of a property for each of predetermined property items from at least one image; and deriving, for each of the property items, a description score indicating a degree of recommendation for including a description regarding the property item in a document based on the property score corresponding to the property item.

According to an eleventh aspect of the present disclosure, there is provided an information processing program for causing a computer to execute a process comprising: deriving a property score indicating a prominence of a property for each of predetermined property items from at least one image; and deriving, for each of the property items, a description score indicating a degree of recommendation for including a description regarding the property item in a document based on the property score corresponding to the property item.

According to the above aspects, the information processing apparatus, information processing method, and information processing program of the present disclosure can support the creation of documents such as interpretation reports.

### BRIEF DESCRIPTION OF THE DRAWINGS

FIG. 1 is a diagram showing an example of a schematic configuration of a medical information system according to an exemplary embodiment.

FIG. 2 is a block diagram showing an example of a hardware configuration of an information processing apparatus according to an exemplary embodiment.

FIG. 3 is a block diagram showing an example of a functional configuration of the information processing apparatus according to an exemplary embodiment.

FIG. 4 is a diagram schematically showing a medical image.

FIG. 5 is a diagram for describing a property score and a description score.

FIG. 6 is a diagram showing an example of a screen for creating an interpretation report.

FIG. 7 is a flowchart showing an example of information processing according to an exemplary embodiment.

FIG. 8 is a diagram showing an example of a trained model that outputs a property score and a description score.

FIG. 9 is a diagram showing an example of a trained model that outputs a property score and a description score.

### DETAILED DESCRIPTION

Hereinafter, each exemplary embodiment of the present disclosure will be described with reference to the drawings.

### First Exemplary Embodiment

First, a configuration of a medical information system 1 to which an information processing apparatus of the present disclosure is applied will be described.

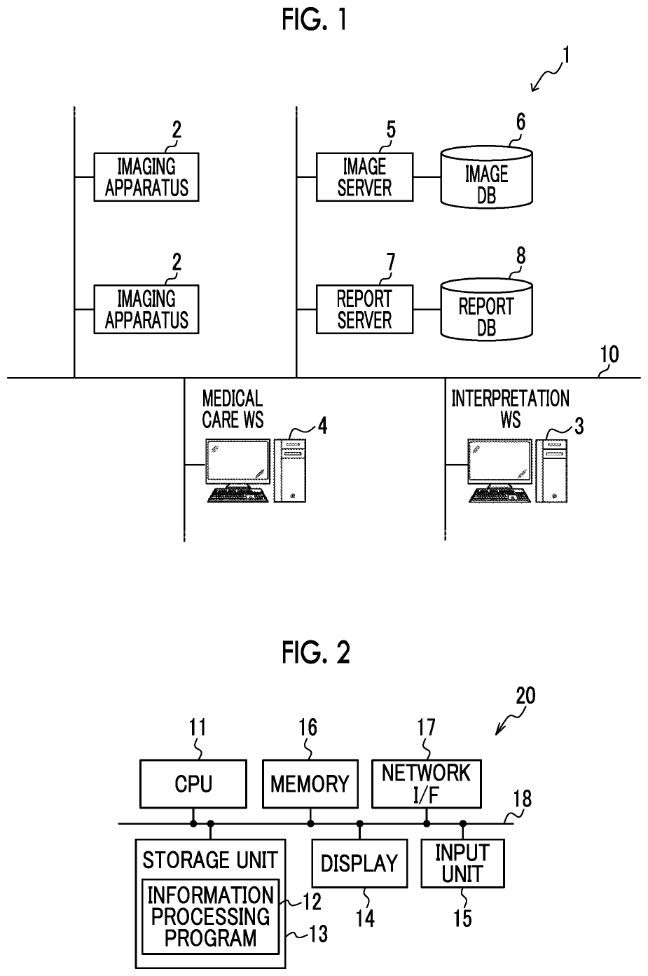

FIG. 1 is a diagram showing a schematic configuration of the medical information system 1. The medical information system 1 shown in FIG. 1 is, based on an examination order from a doctor in a medical department using a known ordering system, a system for imaging an examination target part of a subject, storing a medical image acquired by the imaging, interpreting the medical image by a radiologist and creating an interpretation report, and viewing the interpretation report and observing the medical image to be interpreted in detail by the doctor in the medical department that is a request source.

As shown in FIG. 1, the medical information system 1 is configured to include a plurality of imaging apparatuses 2, a plurality of interpretation work stations (WS) 3 that are interpretation terminals, a medical care WS 4, an image server 5, an image database (DB) 6, a report server 7, and a report DB 8, which are connected via a wired or wireless network 10 so as to be able to communicate with each other.

Each apparatus is a computer on which an application program for causing each apparatus to function as a component of the medical information system 1 is installed. The application program is recorded on a recording medium, such as a digital versatile disc (DVD) or a compact disc read only memory (CD-ROM), and distributed, and is installed on the computer from the recording medium. Alternatively, the application program is stored in a storage apparatus of a server computer connected to the network 10 or in a network storage in a state in which it can be accessed from the outside, and is downloaded and installed on the computer in response to a request.

The imaging apparatus 2 is an apparatus (modality) that generates a medical image showing a diagnosis target part of the subject by imaging the diagnosis target part. Specifically, examples of the imaging apparatus include a simple X-ray imaging apparatus, a CT apparatus, an MRI apparatus, a positron emission tomography (PET) apparatus, and the like. The medical image generated by the imaging apparatus 2 is transmitted to the image server 5 and is saved in the image DB 6.

The interpretation WS 3 is a computer used by, for example, a radiologist of a radiology department to interpret a medical image and to create an interpretation report, and encompasses an information processing apparatus 20 (which will be described in detail later) according to the present exemplary embodiment. In the interpretation WS 3, a viewing request for a medical image to the image server 5, various image processing for the medical image received from the image server 5, display of the medical image, and input reception of comments on findings regarding the medical image are performed. In the interpretation WS 3, an analysis process for medical images, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by, for example, a doctor in a medical department to observe an image in detail, view an interpretation report, create an electronic medical record, and the like, and is configured to include a processing apparatus, a display apparatus such as a display, and an input apparatus such as a keyboard and a mouse. In the medical care WS 4, a viewing request for the image to the image server 5, display of the image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 comprises a storage in which the image DB 6 is configured. This storage may be a hard disk apparatus connected to the image server 5 by a data bus, or may be a disk apparatus connected to a storage area network (SAN) or a network attached storage (NAS) connected to the network 10. In a case where the image server 5 receives a request to register a medical image from the imaging apparatus 2, the image server 5 prepares the medical image in a format for a database and registers the medical image in the image DB 6.

Image data of the medical image acquired by the imaging apparatus 2 and accessory information are registered in the image DB 6. The accessory information includes, for example, an image identification (ID) for identifying each medical image, a patient ID for identifying a subject, an examination ID for identifying an examination, a unique ID (unique identification (UID)) allocated for each medical image, examination date and examination time at which a medical image is generated, the type of imaging apparatus used in an examination for acquiring a medical image, patient information such as the name, age, and gender of a patient, an examination part (an imaging part), imaging information (an imaging protocol, an imaging sequence, an imaging method, imaging conditions, the use of a contrast medium, and the like), and information such as a series number or a collection number in a case where a plurality of medical images are acquired in one examination.

In addition, in a case where the viewing request from the interpretation WS 3 and the medical care WS 4 is received through the network 10, the image server 5 searches for a medical image registered in the image DB 6 and transmits the searched for medical image to the interpretation WS 3 and to the medical care WS 4 that are request sources.

The report server 7 incorporates a software program for providing a function of a database management system to a general-purpose computer. In a case where the report server 7 receives a request to register the interpretation report from the interpretation WS 3, the report server 7 prepares the interpretation report in a format for a database and registers the interpretation report in the report DB 8.

In the report DB 8, an interpretation report including at least the comments on findings created by the radiologist using the interpretation WS 3 is registered. The interpretation report may include, for example, information such as a medical image to be interpreted, an image ID for identifying the medical image, a radiologist ID for identifying the radiologist who performed the interpretation, a lesion name, lesion position information, a property score, and a description score (which will be described in detail later).

Further, in a case where the report server 7 receives the viewing request for the interpretation report from the interpretation WS 3 and the medical care WS 4 through the network 10, the report server 7 searches for the interpretation report registered in the report DB 8, and transmits the searched for interpretation report to the interpretation WS 3 and to the medical care WS 4 that are request sources.

The network 10 is a wired or wireless local area network that connects various apparatuses in a hospital to each other. In a case where the interpretation WS 3 is installed in another hospital or clinic, the network 10 may be configured to connect local area networks of respective hospitals through the Internet or a dedicated line.

Next, the information processing apparatus 20 according to the present exemplary embodiment will be described.

First, with reference to FIG. 2, a hardware configuration of the information processing apparatus 20 according to the present exemplary embodiment will be described. As shown in FIG. 2, the information processing apparatus 20 includes a central processing unit (CPU) 11, a non-volatile storage unit 13, and a memory 16 as a temporary storage area. Further, the information processing apparatus 20 includes a display 14 such as a liquid crystal display and an organic electro luminescence (EL) display, an input unit 15 such as a keyboard and a mouse, and a network interface (I/F) 17 connected to the network 10. The CPU 11, the storage unit 13, the display 14, the input unit 15, the memory 16, and the network I/F 17 are connected to a bus 18. The CPU 11 is an example of a processor in the present disclosure.

The storage unit 13 is realized by a storage apparatus such as a hard disk drive (HDD), a solid state drive (SSD), and a flash memory. An information processing program 12 is stored in the storage unit 13 as the storage medium. The CPU 11 reads out the information processing program 12 from the storage unit 13, loads the read-out program into the memory 16, and executes the loaded information processing program 12.

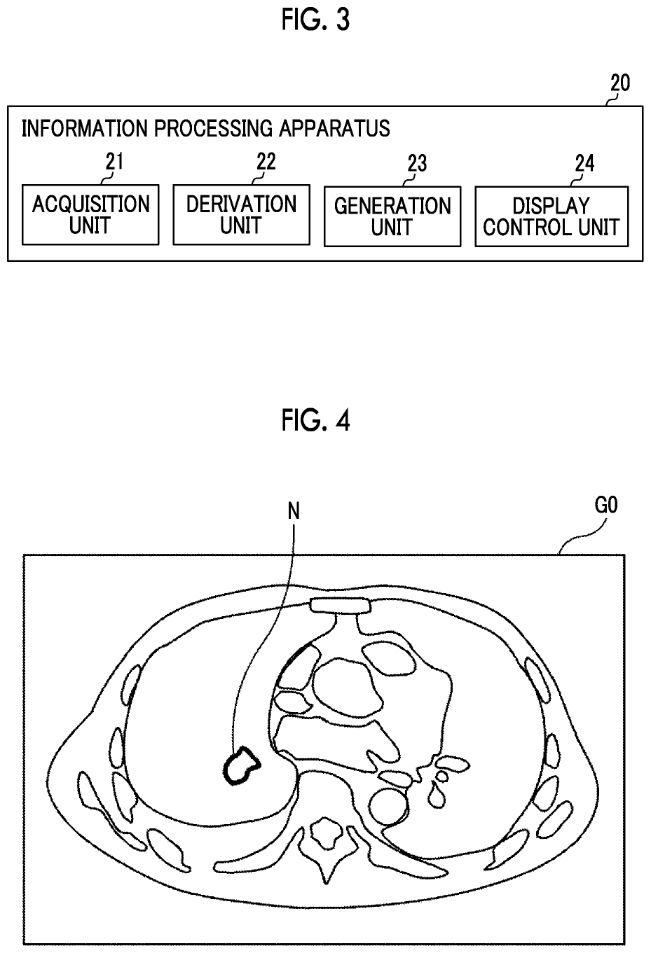

Next, with reference to FIGS. 3 to 6, a functional configuration of the information processing apparatus 20 according to the present exemplary embodiment will be described. As shown in FIG. 3, the information processing apparatus 20 includes an acquisition unit 21, a derivation unit 22, a generation unit 23, and a display control unit 24. The CPU 11 executing the information processing program 12 functions as the acquisition unit 21, the derivation unit 22, the generation unit 23, and the display control unit 24.

The acquisition unit 21 acquires a medical image G0 as an example of the image from the image server 5 via the network I/F 17. FIG. 4 is a diagram schematically showing the medical image G0. In the present exemplary embodiment, as an example, a CT image of a lung is used as the medical image G0. The medical image G0 includes a nodular shadow N as an example of a structure of interest such as a lesion.

Incidentally, from the nodular shadow N, it is possible to grasp the properties of a plurality of property items such as the shape of the margin and the absorption value (density). Therefore, in a case where the radiologist creates an interpretation report on the nodular shadow N, it is necessary to determine which of the description regarding the property item should be described in the interpretation report. For example, it may be determined that the description regarding the property item in which the property is remarkably shown is included in the interpretation report, and the description regarding the property item in which the property is not remarkably shown is not included in the interpretation report. Further, for example, it may be determined that the description regarding a specific property item is included or not included in the interpretation report regardless of the property. It is desired to support the determination as to which of the description regarding the property item should be included in the interpretation report.

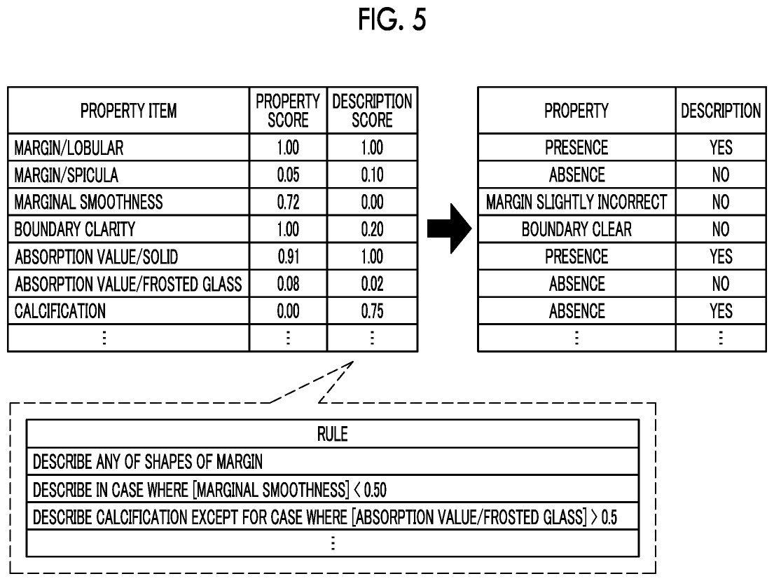

Therefore, the derivation unit 22 according to the present exemplary embodiment derives the property score and the description score in order to support the determination as to which of the description regarding the property item should be included in the interpretation report. FIG. 5 shows an example of a property score and a description score for each predetermined property item related to the nodular shadow N, which is derived from the medical image G0 including the nodular shadow N by the derivation unit 22. FIG. 5 illustrates the shape of the margin (lobular or spicula), marginal smoothness, boundary clarity, absorption value (solidity or frosted glass), and presence or absence of calcification, as the property items related to the nodular shadow N. In FIG. 5, the property score is a value in which the maximum value is 1 and the minimum value is 0, and shows the more remarkable the property in the nodular shadow N as the value is closer to 1. The description score is a value in which the maximum value is 1 and the minimum value is 0, and shows the higher degree of recommendation for including the description regarding the property item in the document as the value is closer to 1.

The derivation unit 22 derives a property score indicating the prominence of the property for each of predetermined property items from at least one medical image G0. Specifically, the derivation unit 22 analyzes the medical image G0 via CAD or the like, specifies the position, type, and size of the structures such as lesions included in the medical image G0, and derives a property score for a property of a predetermined property item related to the specified lesion. That is, the property item is, for example, an item that is predetermined and stored in the storage unit 13 according to at least one of the position, type, or size of the lesion.

In addition, the derivation unit 22 derives, for each of the property items, a description score indicating the degree of recommendation for including the description regarding the property item in the document. Specifically, the derivation unit 22 derives the description score based on a predetermined rule as to whether or not to include the description regarding the property item in the document. The derivation unit 22 may derive the description score according to the degree of conformity with the rule stored in the storage unit 13 in advance, or may derive the description score using a trained model (details will be described later) trained such that the description score is output according to the degree of conformity with the rule.

The rules used by the derivation unit 22 for deriving the description score will be described with reference to a specific example. The “calcification” in FIG. 5 is often used to determine whether the nodular shadow N is malignant or benign. Therefore, the derivation unit 22 may derive a high description score for the property item of “calcification” regardless of the property score.

Further, the derivation unit 22 may derive a description score for each property item based on the property score corresponding to the property item. For example, the derivation unit 22 may derive a description score such that the positive property item is included in the document and the negative property item is not included in the document. Further, as shown in the “margin/lobular” and “margin/spicula”, “absorption value/solid” and “absorption value/frosted glass” in FIG. 5, the derivation unit 22 may derive a description score such that the description score of the property item having a higher property score is high for the same property item.

Further, in the “marginal smoothness” and “boundary clarity” in FIG. 5, it is suspected that the nodular shadow N is malignant as the margin is incorrect and the boundary is unclear. That is, the higher the property score, the more likely it is that the “marginal smoothness” and “boundary clarity” are benign, and the need to dare to describe them in the interpretation report is low. Therefore, as shown in FIG. 5, the derivation unit 22 may derive the description score low in a case where the property scores of “marginal smoothness” and “boundary clarity” are high.

Further, the derivation unit 22 may derive the description score for any of the property items based on the property score derived for any of the other property items. For example, in a case where the nodular shadow N is frosted glass-like, calcification is usually not found, so that the description regarding calcification can be omitted. Therefore, for example, in a case where it is determined from the property score of “absorption value/frosted glass” that the nodular shadow N is likely to be frosted glass-like, by setting the description score of “calcification” to 0.00, the description regarding the property item of calcification may be omitted.

The specific example described above is an example, and the rules are not limited thereto. Further, as the rule, a rule selected by the user from the predetermined rules may be used. For example, prior to the derivation of the description score performed by the derivation unit 22, a screen provided with a check box for selecting any rule from a plurality of predetermined rules may be displayed on the display 14 and the selection by the user may be received.

Based on the description score derived as described above, the generation unit 23 generates a character string for the property item determined to be described in the document with respect to the medical image G0. For example, the generation unit 23 generates comments on findings including a description regarding a property item whose description score is equal to or higher than a predetermined threshold value. As a method of generating the comments on findings by using the generation unit 23, for example, a fixed form for each property item may be used, or a learning model in which machine learning is performed, such as the recurrent neural network described in JP2019-153250A may be used. The character string generated by the generation unit 23 is not limited to the comments on findings, and may be a keyword or the like indicating the property of the property item. Further, the generation unit 23 may generate both the comments on findings and the keyword, or may generate a plurality of comment-on-findings candidates having different expressions.

Further, the generation unit 23 may generate a character string related to a predetermined number of the property items selected in the order of the description scores. For example, in a case where the generation unit 23 generates a character string related to three property items selected in descending order of the description scores, in the example of FIG. 5, a character string related to the property items of “margin/lobular”, “absorption value/solid”, and “calcification” is generated. Further, the user may be able to set the number of property items included in the character string.

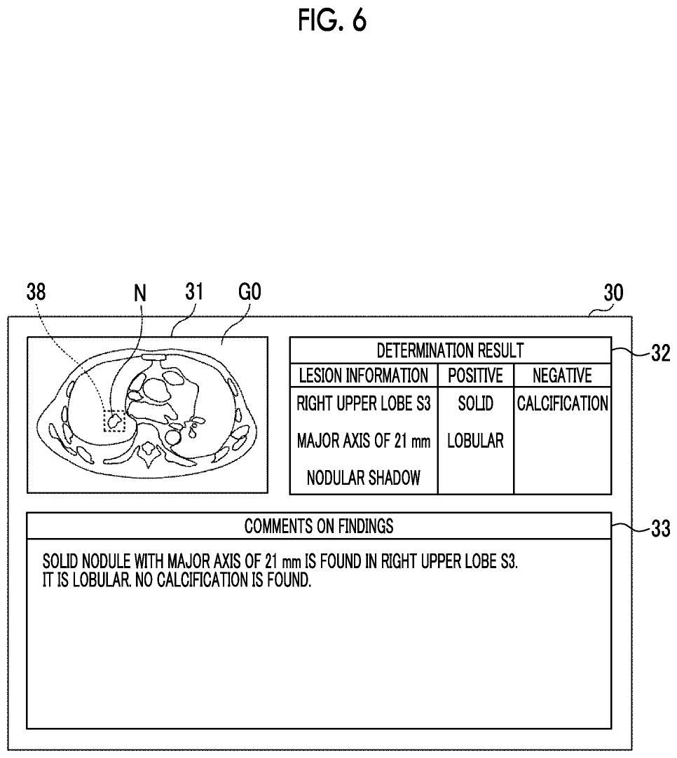

The display control unit 24 performs control such that the character string generated by the generation unit 23 is displayed on the display. FIG. 6 is a diagram showing an example of the interpretation report creation screen 30 displayed on the display 14. The creation screen 30 includes an image display region 31 on which the medical image G0 is displayed, a keyword display region 32 in which a keyword indicating the property of the property item generated by the generation unit 23 is displayed, and a comments-on-findings display region 33 in which comments on findings generated by the generation unit 23 are displayed.

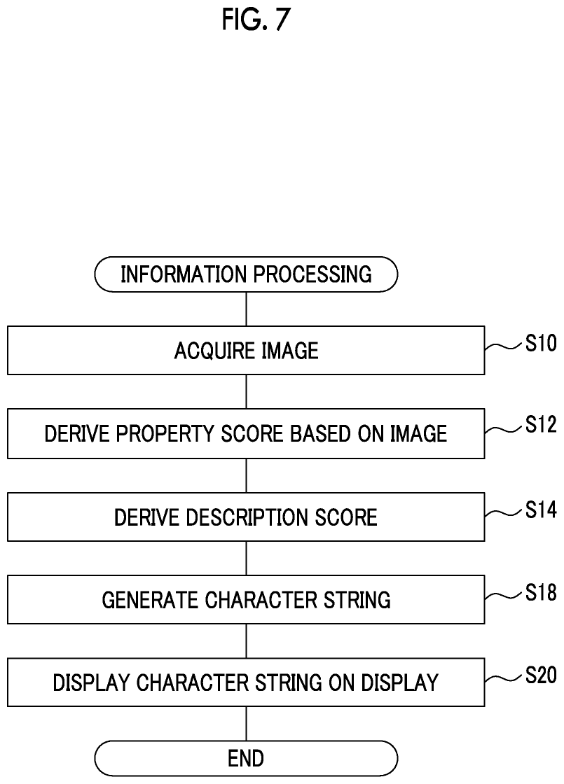

Next, with reference to FIG. 7, operations of the information processing apparatus 20 according to the present exemplary embodiment will be described. The CPU 11 executes the information processing program 12, and thus, the information processing shown in FIG. 7 is executed. The information processing shown in FIG. 7 is executed, for example, in a case where an instruction to start creating an interpretation report for the medical image G0 is input via the input unit 15.

In Step S10 of FIG. 7, the acquisition unit 21 acquires the medical image G0 from the image server 5. In Step S12, the derivation unit 22 specifies the position, type, and size of the lesion included in the medical image G0 based on the medical image G0 acquired in Step S10, and derives a property score for a predetermined property item related to the specified lesion. In Step S14, the derivation unit 22 derives the description score for each property item. In Step S16, the generation unit 23 generates a character string related to the medical image G0 based on the description score. In Step S18, the display control unit 24 performs control such that the character string generated in Step S16 is displayed on the display 14, and ends the process.

As described above, with the information processing apparatus 20 according to the exemplary embodiment of the present disclosure, a property score indicating the prominence of the property for each of predetermined property items is derived from at least one image, and a description score indicating a degree of recommendation for including a description regarding the property item in a document is derived for each of the property items. Since it is possible to grasp the property items that are recommended to be included in the document from such a description score, it is possible to support the determination as to which of the description regarding the property item should be included in the interpretation report, and to support the creation of a document such as an interpretation report.

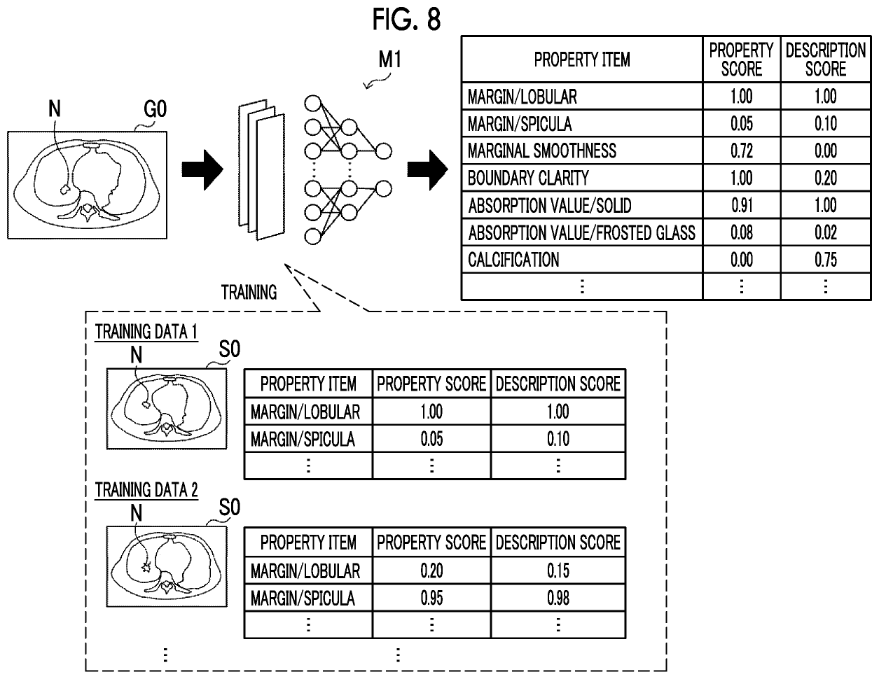

As shown in FIG. 8, the derivation unit 22 may derive the property score and the description score for each property item by inputting the medical image G0 into a trained model M1. The trained model M1 can be realized by machine learning using a model such as a convolutional neural network (CNN), which inputs the medical image G0 and outputs the property score and the description score. The trained model M1 is trained by machine learning using a plurality of combinations of a training image S0 and the property score and the description score derived from the training image S0 as training data.

As the training data, the data in which the radiologist determines the property score and the description score for each property item can be used, for example, for the training image S0 which is a medical image including the nodular shadow N captured in the past. FIG. 8 shows, as an example, a plurality of pieces of training data consisting of a combination of a training image S0 and a property score and a description score scored by the radiologist in the range of 0.00 to 1.00 for the training image S0.

In addition, as the training data created by the radiologist, in addition to the numerically scored property score and description score, the data in which the prominence of the property and the degree of recommendation of description are classified into two or more may be used. For example, instead of the property score for training, information indicating whether or not the property of the property item is found may be used. Further, for example, instead of the description score for training, information indicating the description required/description possible/description unnecessary may be used for the description regarding the property item.

By using the trained model M1 by the derivation unit 22, it is possible to derive a description score in line with the tendency of the training data. Therefore, it is possible to support creation of a document such as an interpretation report.

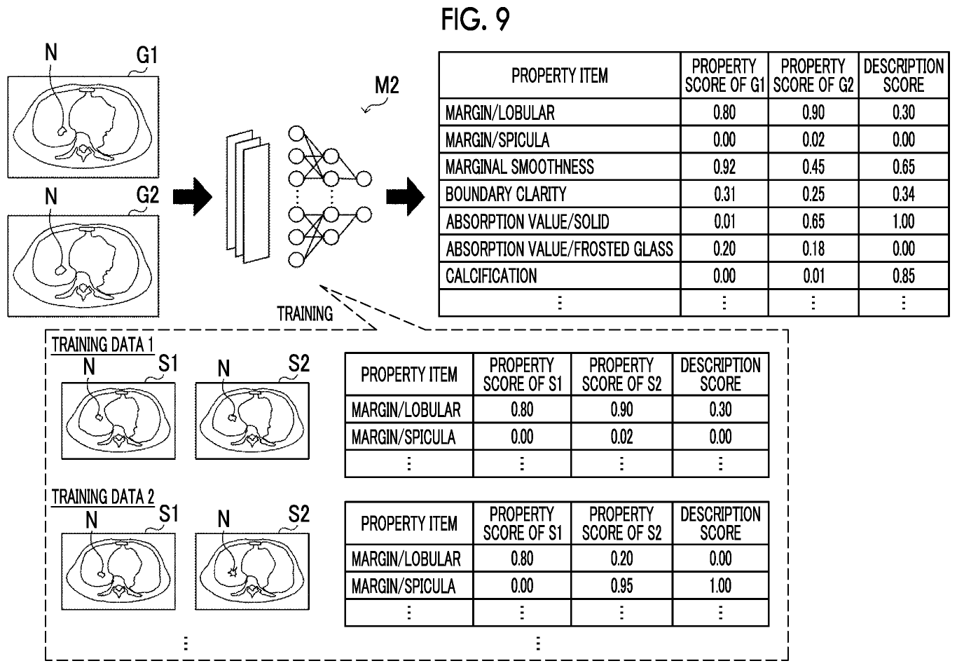

Further, the interpretation report may describe how the properties of the same nodular shadow N have changed over time. Therefore, the derivation unit 22 may derive a property score for each of the plurality of images acquired at different points in time, and may derive a description score for each property item. Here, the derivation unit 22 may derive the description score according to the degree of conformity with the rule stored in the storage unit 13 in advance, or may derive the description score using a trained model M2 shown in FIG. 9, which has been trained such that the description score is output according to the degree of conformity with the rule.

In such a form, a rule defined to derive a description score such that the larger the difference between property scores of a first image G1 and a second image G2, the higher the degree of recommendation for description in the document can be applied. By deriving the description score by the derivation unit 22 based on such a rule, for example, it is possible to grasp the change over time in the past and present properties of the same nodular shadow N.

FIG. 9 shows the trained model M2 that inputs the first image G1 acquired at the first point in time and the second image G2 acquired at the second point in time different from the first point in time, and outputs the property score for each of the first image G1 and the second image G2 and the description score. The trained model M2 can be realized by machine learning using a model such as CNN. The trained model M2 is trained by machine learning using a plurality of combinations of training images S1 and S2 acquired at different points in time and the property score and the description score derived from the training images S1 and S2 as training data.

As the training data, the data in which the radiologist determines the property score and the description score for each property item can be used, for example, for each of the training images S1 and S2 which are medical images captured in two steps for the same nodular shadow N. FIG. 9 shows, as an example, a plurality of pieces of training data consisting of a combination of training images S1 and S2 and a property score and a description score scored by the radiologist in the range of 0.00 to 1.00 for the training images S1 and S2. In addition, as the training data created by the radiologist, in addition to the property score and the description score, the data in which the prominence of the property and the degree of recommendation of description are classified into two or more may be used.

According to such a form, for example, for a property item having a large difference in property scores derived from each of a plurality of images, the description score can be derived such that the degree of recommendation for description in the document is high. Therefore, since it is possible to preferentially describe the property items that change over time in the interpretation report, it is possible to support the creation of a document such as an interpretation report.

In the trained model M2, in a case where the property score and the description score have already been derived for the first image G1 and stored in the report DB 8, instead of the first image G1, the property score and the description score of the first image G1 stored in the report DB 8 may be input.

Further, the trained models M1 and M2 may be trained in advance. In this case, in a case where each of a plurality of radiologists creates an interpretation report for the nodular shadow N having similar properties, since a similar description score can be derived, the description content can be made uniform.

Further, the trained models M1 and M2 may be in a form in which the radiologist who creates the interpretation report creates training data and trains the trained models M1 and M2. In this case, it is possible to derive a description score according to the preference of the radiologist who creates the interpretation report.

Further, in a case where the radiologist corrects the content of the character string generated by the generation unit 23, the trained models M1 and M2 may be retrained using the content of the corrected character string as training data. In this case, even if the radiologist does not dare to create the training data, the description score suitable for the preference of the radiologist can be derived as the interpretation report is created.

In addition, the training data in the trained models M1 and M2 may be data created based on a predetermined rule as to whether or not to include a description regarding a property item in a document. For example, as training data, data in which the property score of “absorption value/frosted glass” is 0.50 or more and the description score of “calcification” is 0.00 is used, and the training model is trained. Then, in a case where the nodular shadow N is likely to be frosted glass-like, the training model can be trained to omit the description regarding the property item of calcification. In this way, by training the training model with the training data created based on the predetermined rule as to whether or not to include the description regarding the property item in the document, the description score derived by the derivation unit 22 can also be obtained in accordance with the predetermined rule.

In addition, the trained models M1 and M2 may be composed of a plurality of models for deriving the property score and the description score, respectively. For example, the trained models M1 and M2 may be composed of a first CNN which uses the medical image G0 as an input and the property score as an output and a second CNN which uses at least one of the medical image G0 or the property score as an input and the description score as an output. That is, the description score may be derived based on the property score instead of the medical image G0, or may be derived based on both the medical image G0 and the property score.

The first CNN is trained by machine learning using, for example, a plurality of combinations of a training image S0 and a property score derived from the training image S0 as training data. The second CNN is trained by machine learning using, for example, a plurality of combinations of a property score derived by the first CNN and a description score derived based on the property score as training data. As an example of the training data of the second CNN, data in which the property score of “absorption value/frosted glass” is 0.50 or more and the description score of “calcification” is 0.00 can be mentioned.

Further, in the above exemplary embodiment, the present disclosure is applied in the case where the interpretation report is created as a document and the comments on findings and the keyword are generated as a character string, but the present disclosure is not limited thereto. For example, the present disclosure may be applied in the case of creating a medical document other than an interpretation report, such as an electronic medical record and a diagnosis report, and other documents including a character string related to an image.

Further, although the various processes are performed using the medical image G0 with a lung as the diagnosis target in the above exemplary embodiments, the diagnosis target is not limited to the lung. In addition to the lung, any part of a human body such as a heart, liver, brain, and limbs can be diagnosed. Further, although various processes are performed using one medical image G0 in the above exemplary embodiments, various processes may be performed using a plurality of images such as a plurality of tomographic images relating to the same diagnosis target.

Further, although the derivation unit 22 specifies the position of the lesion included in the medical image G0 in the above exemplary embodiments, the present disclosure is not limited thereto. For example, the user may select a region of interest in the medical image G0 via the input unit 15, and the derivation unit 22 may determine the properties of the property items of the lesion included in the selected region. According to such a form, for example, even in a case where one medical image G0 includes a plurality of lesions, it is possible to support creation of comments on findings for the lesion desired by the user.

Further, in the above exemplary embodiments, the display control unit 24 may generate an image in which a mark indicating the position of the lesion specified by the derivation unit 22 is added to the medical image G0. In the example of FIG. 6, the nodular shadow N included in the medical image G0 is surrounded by a broken-line rectangle mark 38. This makes it easier, for example, for a reader of the interpretation report to see the region in the image that is the basis of the lesion, without the need for the radiologist to provide comments on findings related to the position of the lesion. Therefore, it is possible to support creation of a document such as an interpretation report. The mark 38 indicating the positions of the lesion is not limited to the broken-line rectangle, but may be various marks such as, for example, a polygon, a circle, an arrow, or the like, and the line type of the mark (a solid line, a broken line, and a dotted line), line color, line thickness, or the like may be changed as appropriate.

Further, in the above exemplary embodiments, each process of the derivation unit 22 and the generation unit 23 in the information processing apparatus 20 encompassed in the interpretation WS 3 may be performed by an external device, for example, another analysis server connected to the network 10. In this case, the external device acquires the medical image G0 from the image server 5, and derives a property score indicating the prominence of the property for each of predetermined property items from the medical image G0. Further, for each property item, a description score indicating the degree of recommendation for including the description of the property item in the document is derived. Further, a character string related to the medical image G0 is generated based on the description score. In the information processing apparatus 20, the display control unit 24 controls the display content to be displayed on the display 14 based on the property score and the description score derived from the external device and the character string generated by the external device.

In the above exemplary embodiments, for example, as hardware structures of processing units that execute various kinds of processing, such as the acquisition unit 21, the derivation unit 22, the generation unit 23, and the display control unit 24, various processors shown below can be used. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (programs).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor. As an example where a plurality of processing units are configured by one processor, first, there is a form in which one processor is configured by a combination of one or more CPUs and software as typified by a computer, such as a client or a server, and this processor functions as a plurality of processing units. Second, there is a form in which a processor for realizing the function of the entire system including a plurality of processing units via one integrated circuit (IC) chip as typified by a system on chip (SoC) or the like is used. In this way, various processing units are composed of one or more of the above-described various processors as hardware structures.

Furthermore, as the hardware structure of the various processors, more specifically, an electrical circuit (circuitry) in which circuit elements such as semiconductor elements are combined can be used.

The disclosure of JP2020-036290 filed on Mar. 3, 2020 is incorporated herein by reference in its entirety. All literatures, patent applications, and technical standards described herein are incorporated by reference to the same extent as if the individual literature, patent applications, and technical standards were specifically and individually stated to be incorporated by reference.

What is claimed is:

1. An information processing apparatus comprising at least one processor, wherein the at least one processor is configured to:
derive a property score indicating a prominence of a property for each of predetermined property items from at least one image; and
derive, for each of the property items, a description score indicating a degree of recommendation for including a description regarding the property item in a document.

2. The information processing apparatus according to claim 1, wherein the at least one processor is configured to derive the description score based on a predetermined rule as to whether or not to include the description regarding the property item in the document.

3. The information processing apparatus according to claim 1, wherein the at least one processor is configured to derive the description score for each property item based on the property score corresponding to the property item.

4. The information processing apparatus according to claim 1, wherein the at least one processor is configured to derive the description score for any of the property items based on the property score derived for any of the other property items.

5. The information processing apparatus according to claim 1, wherein:
the at least one processor is configured to input the image into a trained model to derive the property score and the description score, and
the trained model is a model that is trained by machine learning using a plurality of combinations of a training image, and the property score and the description score derived from the training image as training data, inputs the image, and outputs the property score and the description score.

6. The information processing apparatus according to claim 1, wherein the at least one processor is configured to:
derive the property score for each of a plurality of the images acquired at different points in time, and
derive the description score for each property item.

7. The information processing apparatus according to claim 1, wherein the at least one processor is configured to derive the property score based on at least one of a position, type, or size of a structure included in the image.

8. The information processing apparatus according to claim 1, wherein the at least one processor is configured to:
generate a character string related to the image based on the description score; and
perform control such that the character string is displayed on a display.

9. The information processing apparatus according to claim 1, wherein the at least one processor is configured to generate a character string related to a predetermined number of the property items selected in an order of the description scores.

10. An information processing method comprising:
deriving a property score indicating a prominence of a property for each of predetermined property items from at least one image; and
deriving, for each of the property items, a description score indicating a degree of recommendation for including a description regarding the property item in a document based on the property score corresponding to the property item.

11. A non-transitory computer-readable storage medium storing an information processing program for causing a computer to execute a process comprising:
deriving a property score indicating a prominence of a property for each of predetermined property items from at least one image; and
deriving, for each of the property items, a description score indicating a degree of recommendation for including a description regarding the property item in a document based on the property score corresponding to the property item.
